(19) United States
(12) Patent Application Publication
(10) Pub. No.: US 2016/0346511 A1
(43) Pub. Date: Dec. 1, 2016

(54) MIDDLE EAR SURGICAL ATTACHMENTS FOR SUCTION CATHETERS AND SUCTION CATHETERS INCLUDING THE SAME

(71) Applicant: Webb Medical LLC, Philadelphia, PA (US)

(72) Inventors: Michael Cohen, Newton Center, MA (US); Steven Krupnick, Philadelphia, PA (US)

(21) Appl. No.: 14/848,987

(22) Filed: Sep. 9, 2015

Related U.S. Application Data
(60) Provisional application No. 62/169,149, filed on Jun. 1, 2015.

(51) Int. Cl. A61M 25/00; A61M 1/00; A61F 11/00; A61B 1/04; A61B 1/227

(52) U.S. Cl. CPC A61M 25/0082; A61B 1/04; A61B 1/227; A61F 11/004; A61M 1/008; A61M 2202/0413

(57) ABSTRACT

A medical attachment for use on the free end of a suction catheter extending into the middle ear of a patient is disclosed. The attachment can be part of a kit of plural attachments, each attachment being configured for accomplishing a different task. Each attachment includes a proximal base portion and an angularly extending distal portion terminating in a working tip. The working tip can be a foam member, a spade shaped member, or a pointed member. The base portion serves to releasably mount the attachment on the free end of the suction catheter. Also disclosed is a specially constructed suction catheter including a tip in the form of a foam member, a spade shaped member, or a pointed tip member.

### CROSS-REFERENCE TO RELATED APPLICATIONS

This utility application claims the benefit under 35 U.S.C. §119(e) of Provisional Application Ser. No. 62/169,149 filed on Jun. 1, 2015, entitled MIDDLE EAR SURGICAL ATTACHMENTS FOR SUCTION CATHETERS AND SUCTION CATHETERS INCLUDING THE SAME. The entire disclosure of that provisional application is incorporated by reference herein.

### STATEMENT REGARDING FEDERALLY SPONSORED RESEARCH OR DEVELOPMENT

Not Applicable

### INCORPORATION-BY-REFERENCE OF MATERIAL SUBMITTED ON A COMPACT DISK

Not Applicable

### FIELD OF THE INVENTION

The disclosed invention relates to medical devices and more particularly to otological surgical devices for middle ear surgery.

### BACKGROUND OF THE INVENTION

The surgical treatment of conditions in the middle ear is typically accomplished by lifting the eardrum out of the natural groove in which it sits. That process involves making an incision in the skin of the ear canal and basically dissecting the layer between the skin and the underlying bone to lift the eardrum out of its anatomical location and enter the space beyond the eardrum that is known as the middle ear. The otological instruments that are typically used for this process are quite small and have various types of tips, such as spades, points, etc., on the distal end thereof.

Currently middle ear dissection procedures are visualized by the surgeon using either an endoscope extending into the ear canal or by use of a microscope. If the surgeon is using a microscope he/she can make use of both of his/her hands to effect the surgical procedure. If, however, the procedure is to be accomplished endoscopically, that typically requires that the surgeon use one of his/her hands to hold the endoscope (unless it is held by some mechanical support).

In any case the surgical procedure, e.g., tissue dissection, in the middle ear is typically accomplished by the surgeon holding a small cutting or scraping instrument in his/her dominant hand to cut or scrape the tissue while holding a suction catheter, such as a Baron catheter, in the non-dominant hand. This enables the surgeon to use the suction catheter to remove blood and retract and/or elevate tissue during the dissection procedure. That technique makes endoscopic surgery challenging since it necessitates the surgeon using both hands, one for the instrument and the other for the suction catheter. In particular, the surgeon typically places the endoscope in his/her non-dominant hand, while holding the small surgical instrument in his/her dominant hand. As such, the surgeon no longer has suction to clear the field of blood and maintain visualization. Accordingly, the surgeon has to pause from time to time to use the suction catheter to clear the field of blood and to improve visualization.

While endoscope holders are available to hold the endoscope during surgical procedures, and thus eliminate the holding problem an additional problem exists. In particular, the ear canal is too small to accommodate an endoscope, a surgical instrument and a suction catheter while enabling the effective manipulation of all three. This is particularly true if the procedure is to be accomplished on a child, whose ear canal is significantly smaller than an adult's ear canal.

Thus, a need exists for surgical devices to overcome these problems. The subject invention does that by combining the suction instrument and the cutting/scraping instrument into a single instrument that can be used by one hand.

### SUMMARY OF THE INVENTION

In accordance with one aspect of this invention there is provided a medical attachment for use on the free end of a suction catheter extending into the middle ear of a patient, with an endoscope extending into the associated ear canal of the patient. The medical attachment comprises a proximal base portion and an angularly extending distal portion. The angularly extending distal portion terminates in a working tip. The working tip is selected from the group consisting of a foam member, a spade shaped member, and a pointed tip member. The base portion is configured for releasably mounting the medical attachment on the free end of the suction catheter. The working tip is configured to be brought into engagement with tissue in the middle ear to perform a medical procedure thereon by a user manipulating the suction catheter with one hand, with the other hand of the user being free to manipulate the endoscope to permit the viewing of the medical procedure.

In accordance with another aspect of the invention there is provided a kit of medical attachments for use on the free end of a suction catheter extending into the middle ear of a patient, with an endoscope extending into the associated ear canal of the patient. The kit comprises a plurality of medical attachments, each of which comprises a proximal base portion and an angularly extending distal portion. The angularly extending distal portion of each attachment terminates in a working tip. The working tip of one of the attachments comprises a foam member. The working tip of another of the attachments comprises a spade shaped member. The working tip of still another of the attachments comprises a pointed tip member. The base portion of each of the medical attachments is configured for releasably mounting the medical attachment on the free end of the suction catheter. The working tip is configured to be brought into engagement with tissue in the middle ear to perform a medical procedure thereon by a user manipulating the suction catheter with one hand, with the other hand of the user being free to manipulate the endoscope to permit the viewing of the medical procedure.

In accordance with another aspect of the invention there is provided a suction catheter having a free end configured to be extended into the middle ear of a patient, with an endoscope extending into the associated ear canal of the patient. The free end of the suction catheter comprises an integral angularly extending working tip. The working tip is selected from the group consisting of a foam member, a spade shaped member, and a pointed tip member. The working tip is configured to be brought into engagement with tissue in the middle ear to perform a medical procedure thereon by a user manipulating the suction catheter with one hand, with the other hand of the user being free to manipulate the endoscope to permit the viewing of the medical procedure.

### BRIEF DESCRIPTION OF THE DRAWING FIGURES

FIG. 1 is an isometric view of a conventional otological suction catheter, e.g., a Baron catheter, on which one exemplary embodiment of a surgical tip, e.g., a sponge tip, constructed in accordance with this invention is mounted;

FIG. 2 is an enlarged side elevation view of the distal end of the suction catheter shown in FIG. 1 with the exemplary surgical tip mounted thereon;

FIG. 3 is a slightly enlarged end view taken along line 3-3 of FIG. 2;

FIG. 4 is a slightly enlarged cross-sectional view taken along line 4-4 of FIG. 2;

FIG. 5 is a side elevation view, similar to FIG. 2, but showing an alternative exemplary embodiment of a surgical tip, e.g., a spade tip, constructed in accordance with this invention;

FIG. 6 is top plan view taken along line 6-6 of FIG. 5;

FIG. 7 is a slightly enlarged end view taken along line 7-7 of FIG. 5;

FIG. 8 is a side elevation view, similar to FIGS. 2 and 5, but showing still another alternative exemplary embodiment of a surgical tip, e.g., a pointed or pick tip, constructed in accordance with this invention;

FIG. 9 is top plan view taken along line 9-9 of FIG. 8; and

FIG. 10 is a slightly enlarged end view taken along line 10-10 of FIG. 8.

### DETAILED DESCRIPTION OF THE PREFERRED EMBODIMENTS

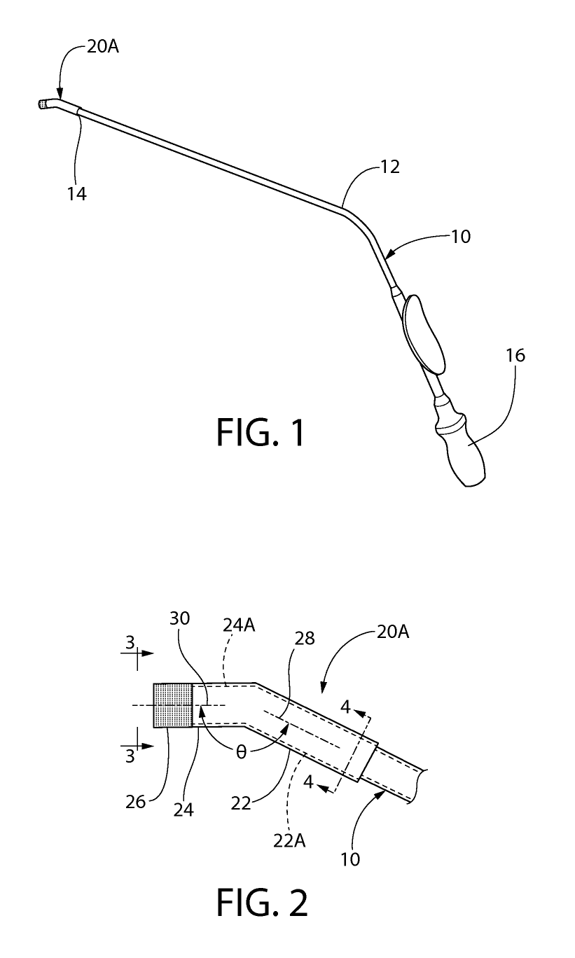

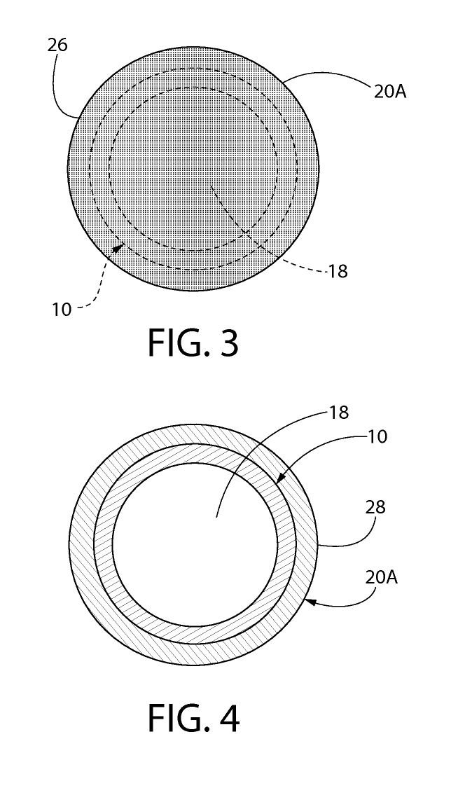

Referring now to the drawings wherein like characters refer to like parts, there is shown in FIG. 1 an exemplary embodiment of a conventional suction catheter 10, e.g., a Baron catheter, on which one exemplary medical attachment 20A constructed in accordance with this invention is mounted. Before describing the medical attachment 20A, a brief description of the catheter 10 is in order. To that end, as can be seen in FIG. 1, the catheter 10 basically comprises an elongated tubular body 12 having a distal end 14 and a proximal end 16. The proximal end 16 includes a handle to enable the suction catheter to be held by a surgeon or other operating personnel. The tubular body is hollow and thus includes a central lumen or passageway 18 (FIG. 4) through which suction is applied from a suction source (not shown) to the distal end 14 of the catheter.

The exemplary attachment 20A is configured for releasable mounting on the distal end 14 of the catheter 10 and includes a working end that is shaped to accomplish a particular otological task. As will be seen from the discussion to follow the attachment 20A is one of several types of attachments that can be constructed in accordance with this invention, with each attachment being configured for a particular otological task. The various attachments can be provided as a kit of replaceable attachments, with each attachment being shaped like a corresponding tip of a typical conventional instrument set. If desired, each attachment may be disposable, so that it can be disposed of after use, or may be non-disposable so that it can be cleaned and disinfected for subsequent reuse. Moreover, it must be pointed out at this juncture, that the subject invention is not limited to attachments for conventional suction catheters, such as that shown in FIG. 1. Thus, the subject invention also contemplates a suction catheter that is specially constructed so that its free end includes a tip constructed in accordance with this invention.

In any case, each of the attachments/tips of this invention (whether replaceable attachments for mounting on a conventional suction catheter, or as an integral tip of a specially constructed suction catheter) includes a working end of a particular construction, shape and size to accomplish a particular otological task.

For example, in FIG. 2 there is shown an attachment 20 in the form of a “sponge” tip. This type of attachment is used to draw blood into the suction catheter without drawing in adjacent tissue to thereby protect that tissue while it is being elevated as an initial step in a typical middle ear dissection procedure (to be described later). The sponge tip 20 basically comprises a proximal base portion 22 and an angularly extending distal portion 24, which are preferably an integral unit and can be formed of any suitable material, e.g., surgical steel. The angularly extending distal portion terminates in a free working end 26. The proximal base 22 portion is in the form of a hollow tubular hub having a central longitudinal axis 28. The hollow tubular hub includes a central passageway 22A that is configured to receive the free end 14 of the suction catheter 10 to releasably mount the attachment 20A thereon. To that end, the diameter passageway 22A of the hub is preferably sized to accommodate the distal end of a conventional catheter therein. As is known those catheters come in 5 French, 20 gauge and 24 gauge sizes. Thus, the inside diameter of the hub's passageway is sized accordingly. The angularly extending distal portion 24 of the attachment 20A is also a hollow tubular section having a central longitudinal axis 30 and a central passageway 24A. The outside and inside diameters of the angularly extending portion 24 are preferably the same as the respective outside and inside diameters as the proximal portion 22, but that need not be the case. Moreover, the angle between the longitudinal axis 28 of the proximal base portion 22 and the longitudinal axis 30 of the angularly extending distal portion 24 is within the range of approximately 160 degrees to approximately 135 degrees, although other angular arrangements are contemplated. Thus, for example the angle between the distal end portion 24 of the tip and its proximal portion 22 may be approximately 160 degrees, or 150 degrees or 135 degrees. Other angles can be used as well. In fact, it is contemplated that the interface between the proximal base portion 22 and an angularly extending distal portion 24 be adjustable so that the angle can be adjusted to any angle desired.

In any case, the hollow interior of the proximal base portion 22 and the hollow interior of angularly extending distal portion 24 are in fluid communication with each other so that suction can be applied through the body of the attachment to the working end 26. In accordance with one preferred exemplary embodiment of this invention, the length of the base portion 22 from its proximal end to the point at which it merges with the angularly extending distal portion 24 is approximately 7 mm, and the length of the angularly extending distal portion 24 from that merger point to the distal end of the angularly extending distal portion 24 is approximately 3 mm. The working tip 26 of the attachment 20A is in the form of a body of open cell foam which is fixedly secured to the distal end of the angularly extending distal portion 24. Preferably the material making up the foam is polyvinyl acetyl, but other open cell foams can be used, if desired. The length of the body of foam is approximately 1-1.5 mm, but other lengths are contemplated.

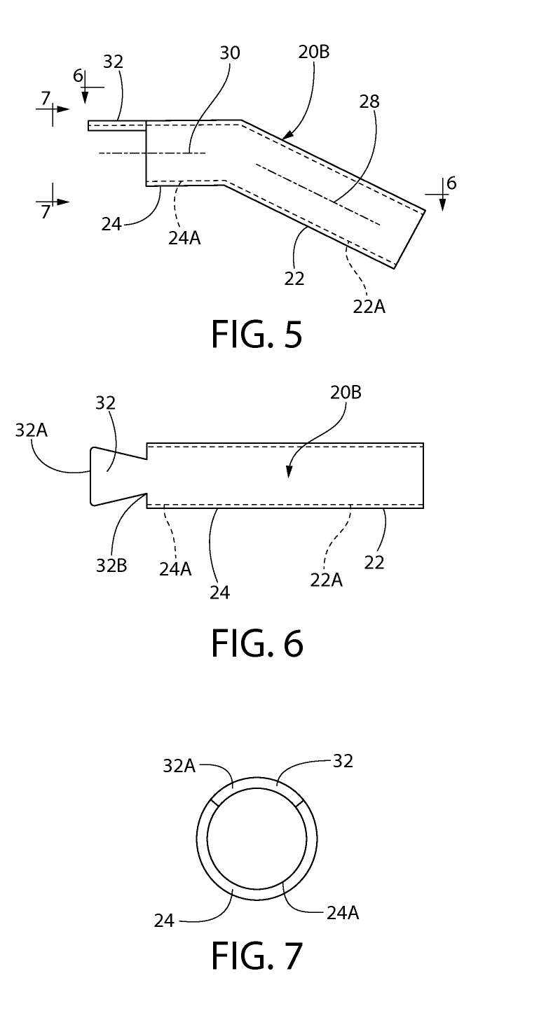

Turning now to FIGS. 5-7, another exemplary embodiment of an attachment 20B constructed in accordance with this invention is shown. The attachment 20B is constructed identically to the sponge attachment 20A except for the working end, which is in the form of a spade tip. In the interest of brevity, the common features of the attachments 20A and 20B will be given the same reference numbers and the details of the construction and operation of those features will not be reiterated. Thus, as can be seen the attachment 20B includes a proximally located base portion 22, an angularly extending distal portion 24 and a spade shaped extension or tip 32 projecting distally from the distal end of the angularly extending distal portion 24 adjacent the top thereof. In fact, the spade tip constitutes an extension of the wall of the tube making up the angularly extending distal portion 24. The spade tip extends over the open end of the angularly extending distal portion 24 and parallel to the central longitudinal axis 30 thereof. In accordance with one exemplary preferred embodiment of a spade attachment 20B, the tip 32 is approximately 1-1.5 mm long. As can be best seen in FIG. 6 the free distal end 32A of the spade tip 32 is wider, e.g., flares outward, from the root 32B of the spade tip. The amount of flare of the spade tip can be whatever is deemed desirable for a particular otological procedure. As will be appreciated by those skilled in the art, the flared spade tip can be used to elevate either skin or diseased tissue from the surface of the middle ear by scraping the spade along the tissue-bone interface while the suction applied through its passageways 22A and 24A removes the blood and debris, thereby improving visualization.

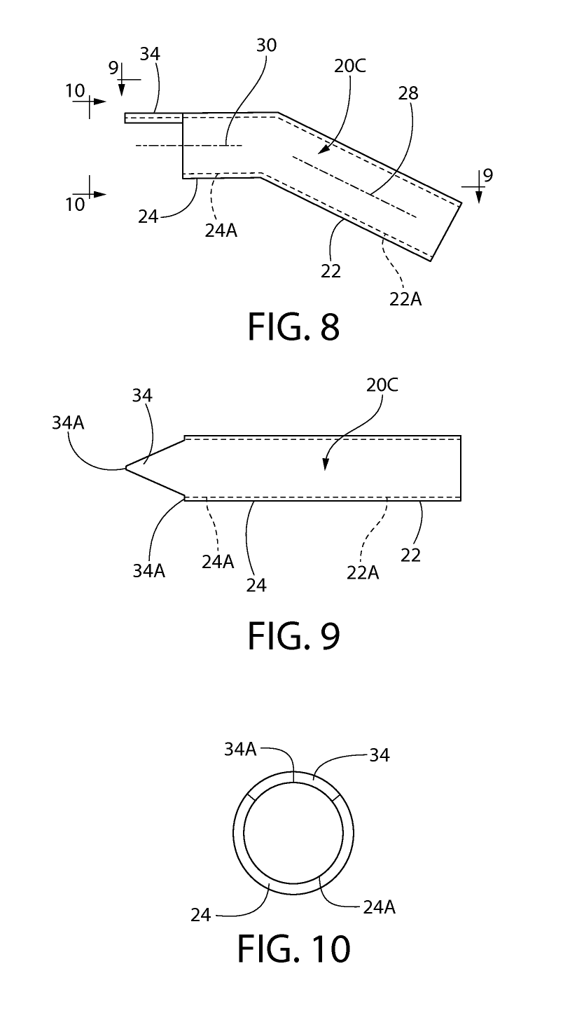

Another type of attachment constructed in accordance with this invention is a “point or pick” tip attachment 20C, like shown in FIGS. 8-10. The attachment 20C is used for very fine dissection, e.g., to take off a layer of very delicate tissue by scraping the point along the tissue-bone interface. The attachment 20C is constructed identically to the spade attachment 20B except for the working end, which is in the form of a pointed or pick tip. In the interest of brevity, the common features of the attachments 20B and 20C will be given the same reference numbers and the details of the construction and operation of those features will not be reiterated. Thus, as can be seen the attachment 20C includes a proximally located base portion 22 and an angularly extending distal portion 24 and a V-shaped pointed extension or tip 34. The tip 34 projects distally from the distal end of the angularly extending distal portion 24 adjacent the top thereof. In fact, the pick tip constitutes an extension of the wall of the tube making up the angularly extending distal portion 24 and extends over the open end of the angularly extending distal portion 24 and parallel to the central longitudinal axis 30 thereof. As can be best seen in FIG. 9 the free distal end 32A of the spade tip 32 is a point. In particular, the tip 34 tapers inward from the root 34B of the tip 34 to the point at the free end thereof. The amount of taper of the pick tip can be whatever is deemed desirable for a particular otological procedure. The length of the tip 34 can be any length deemed desirable. In accordance with one exemplary preferred embodiment of a pick tip attachment 20C the pointed tip is approximately 1-1.5 mm long.

The following constitutes use of the attachments of the subject invention during a cholesteatoma surgery. As is known a cholesteatoma is a cyst of skin that forms in the middle ear space behind the eardrum which must be excised. The first step is to access the middle ear space, which is medial to the eardrum. Thus, the surgeon makes an incision in the skin of the ear canal and lifts up that skin which will take the surgeon to the edge of the eardrum. This initial cutting can be accomplished by a conventional cutting knife, such as a Rosen knife, or an otological round knife Once that has been accomplished the sponge tip attachment 20A can be used on the suction catheter 10 to basically scrape in the plane between the skin of the ear canal and the bone of the ear canal. An endoscope (not shown) may be located in the ear canal to provide visualization of this procedure. The endoscope need not extend through the opening produced by the lifting of the skin into the operative space in the middle ear, since that space will be visible from the ear canal. Thus, the endoscope may remain in the ear canal for the entire surgery.

As the attachment 20A is pushed along the tissue the sponge body at its free end compresses so that the distal end of underlying cylindrical base portion of the tip scan scrape along that interface to gently elevate the skin of the ear canal. This process is known as the elevation of the tympanomeatal flap and exposes the middle ear. Once the cholesteatoma in the middle ear has been exposed the surgeon can then use the spade attachment 20B on the suction catheter 10 for coarser dissection, followed by use of the pick attachment 20C on the suction catheter for finer dissection, alternating as much as desired to remove the cholesteatoma.

As will be appreciated by those skilled in the art the attachments/tips of this invention can be used for other otologic procedures, than the exemplary middle ear dissection described above.

Without further elaboration the foregoing will so fully illustrate my invention that others may, by applying current or future knowledge, adopt the same for use under various conditions of service.

We claim:

1. A medical attachment for use on the free end of a suction catheter extending into the middle ear of a patient and with an endoscope extending into the associated ear canal of the patient, said medical attachment comprising a proximal base portion and an angularly extending distal portion, said angularly extending distal portion terminating in a working tip, said working tip being selected from the group consisting of a foam member, a spade shaped member, an a pointed tip member, said base portion being configured for releasably mounting said medical attachment on the free end of the suction catheter, said working tip being configured to be brought into engagement with tissue in the middle ear to perform a medical procedure thereon by a user manipulating the suction catheter with one hand, with the other hand of the user being free to manipulate the endoscope to permit the viewing of the medical procedure.

2. The medical attachment of claim 1 wherein said proximal base portion comprises a hollow tubular hub having a central longitudinal axis, said hollow tubular hub being configured to receive the free end of the suction catheter to releasably mount said medical attachment thereon, and wherein said angularly extending distal portion of said medical attachment comprises a hollow tubular section having a central longitudinal axis.

3. The medical attachment of claim 2 wherein the angle between said central longitudinal axis of said angularly extending distal portion and said central longitudinal axis of said proximal base portion is in the range of approximately 160 degrees to approximately 135 degrees.

4. The medical attachment of claim 3 wherein the angle between said central longitudinal axis of said angularly extending distal portion and said central longitudinal axis of said proximal base portion is approximately 160 degrees.

5. The medical attachment of claim 3 wherein the angle between said central longitudinal axis of said angularly extending distal portion and said central longitudinal axis of said proximal base portion is approximately 150 degrees.

6. The medical attachment of claim 3 wherein the angle between said central longitudinal axis of said angularly extending distal portion and said central longitudinal axis of said proximal base portion is approximately 135 degrees.

7. The medical attachment of claim 2 wherein said working tip is a foam member, said foam member being open cell foam to enable blood to pass therethrough upon the application of suction to said medical attachment by the suction catheter.

8. The medical attachment of claim 2 wherein said working tip is a spade shaped member projecting distally from said angularly extending distal portion and parallel to said central longitudinal axis of said angularly extending distal portion, said hollow tubular section of said angularly extending distal portion being configured to enable blood to pass therethrough upon the application of suction to said medical attachment by the suction catheter.

9. The medical attachment of claim 2 wherein said working tip is a pointed tip member projecting distally from said angularly extending distal portion and parallel to said central longitudinal axis of said angularly extending distal portion, said hollow tubular section of said angularly extending distal portion being configured to enable blood to pass therethrough upon the application of suction to said medical attachment by the suction catheter.

10. A kit of medical attachments for use on the free end of a suction catheter extending into the middle ear of a patient and with an endoscope extending into the associated ear canal of the patient, said kit comprising a plurality of medical attachments, each of said medical attachments comprising a proximal base portion and an angularly extending distal portion, said angularly extending distal portion terminating in a working tip, said working tip of one of said attachments comprising a foam member, said working tip of another of said attachments comprising a spade shaped member, said working tip of still another of said attachments comprising an a pointed tip member, said base portion of each of said medical attachments being configured for releasably mounting said medical attachment on the free end of the suction catheter, said working tip being configured to be brought into engagement with tissue in the middle ear to perform a medical procedure thereon by a user manipulating the suction catheter with one hand, with the other hand of the user being free to manipulate the endoscope to permit the viewing of the medical procedure.

11. The kit of claim 10 wherein said proximal base portion of each of said medical attachments comprises a hollow tubular hub having a central longitudinal axis, said hollow tubular hub being configured to receive the free end of the suction catheter to releasably mount said medical attachment thereon, and wherein said angularly extending distal portion of each of said medical attachments comprises a hollow tubular section having a central longitudinal axis.

12. The kit of claim 11 wherein the angle between said central longitudinal axis of said angularly extending distal portion and said central longitudinal axis of said proximal base portion is in the range of approximately 160 degrees to approximately 135 degrees.

13. The kit of claim 12 wherein the angle between said central longitudinal axis of said angularly extending distal portion and said central longitudinal axis of said proximal base portion is approximately 160 degrees.

14. The kit of claim 12 wherein the angle between said central longitudinal axis of said angularly extending distal portion and said central longitudinal axis of said proximal base portion is approximately 150 degrees.

15. The kit of claim 12 wherein the angle between said central longitudinal axis of said angularly extending distal portion and said central longitudinal axis of said proximal base portion is approximately 135 degrees.

16. The kit of claim 11 wherein said foam member is open cell foam to enable blood to pass therethrough upon the application of suction to said medical attachment by the suction catheter.

17. The kit of claim 11 wherein said working tip is a spade shaped member projecting distally from said angularly extending distal portion and parallel to said central longitudinal axis of said angularly extending distal portion, said a hollow tubular section of said angularly extending distal portion being configured to enable blood to pass therethrough upon the application of suction to said medical attachment by the suction catheter.

18. The kit of claim 11 wherein said working tip is a pointed tip member projecting distally from said angularly extending distal portion and parallel to said central longitudinal axis of said angularly extending distal portion, said a hollow tubular section of said angularly extending distal portion being configured to enable blood to pass therethrough upon the application of suction to said medical attachment by the suction catheter.

19. A suction catheter having a free end configured to be extended into the middle ear of a patient with an endoscope extending into the associated ear canal of the patient, said free end of said suction catheter comprising an integral angularly extending working tip, said working tip being selected from the group consisting of a foam member, a spade shaped member, and a pointed tip member, said working tip being configured to be brought into engagement with tissue in the middle ear to perform a medical procedure thereon by a user manipulating the suction catheter with one hand, with the other hand of the user being free to manipulate the endoscope to permit the viewing of the medical procedure.

20. The suction catheter of claim 19 wherein said suction catheter comprises a body portion located proximally of said free end, said body portion having a central longitudinal axis, said angularly extending working tip having a longitudinal axis, with the angle between said longitudinal axis of said angularly extending working tip and said central longitudinal axis of said body portion being in the range of approximately 160 degrees to approximately 135 degrees.

21. The suction catheter of claim 20 wherein the angle between said central longitudinal axis of said angularly extending distal portion and said central longitudinal axis of said proximal base portion is approximately 160 degrees.

22. The suction catheter of claim 20 wherein the angle between said central longitudinal axis of said angularly extending distal portion and said central longitudinal axis of said proximal base portion is approximately 150 degrees.

23. The suction catheter of claim 20 wherein the angle between said central longitudinal axis of said angularly extending distal portion and said central longitudinal axis of said proximal base portion is approximately 135 degrees.

24. The suction catheter of claim 20 wherein said working tip is a foam member, said foam member being open cell foam.

25. The suction catheter of claim 20 wherein said working tip is a spade shaped member.

26. The suction catheter of claim 20 wherein said working tip is a pointed tip member.
